(19) United States
(12) Patent Application Publication
(10) Pub. No.: US 2004/0158118 A1
(43) Pub. Date: Aug. 12, 2004

(54) Terminus-spacer component of a string comprising one or more spacer components and one or more implantation seeds

(76) Inventors: Christopher D. Drobnik, Wauconda, IL (US); Michael W. Drobnik, Downers Grove, IL (US); Scott C. Jones, Bloomingdale, IL (US); Frank Lyman, Lake Zurich, IL (US)

Correspondence Address: PATTI & BRILL, ONE NORTH LASALLE STREET, 44TH FLOOR, CHICAGO, IL, 60602, US

(21) Appl. No.: 10/361,210

(22) Filed: Feb. 10, 2003

(51) Int. Cl. A61N 5/00; A61M 36/00

(52) U.S. Cl. 600/8

(57) ABSTRACT

An apparatus in one example comprises a terminus-spacer component that comprises a first end portion and a second end portion. The first end portion is configured to form a coupled connection with an implantation seed. The second end portion serves to terminate a string that comprises one or more spacer components that comprise the terminus-spacer component and one or more implantation seeds that comprise the implantation seed.

### TECHNICAL FIELD

The invention relates generally to implantation seeds and more particularly to spacing of implantation seeds.

### BACKGROUND

Bodily cancers are commonly treated using radiation therapy. Radiation therapy employs high energy radiation to kill cancer cells. One type of radiation therapy is brachytherapy, in which a source of radiation is in direct contact with an afflicted tissue. A common brachytherapy treatment, transperineal seed implantation, involves placing radioactive seeds in the prostate gland to kill prostate gland cancer cells. A physician employs tools, for example, ultrasound, computerized axial tomography (“CAT”) scans, and X-ray images in concert with dose-planning computer software programs to evaluate the medical condition of a patient. The physician constructs an optimal treatment plan to evenly distribute radiation throughout the afflicted tissue. Radioactive seeds of discrete radioactive strengths are inserted through multiple implantation needles at positions in the prostate gland corresponding to the treatment plan. Multiple implantation needles are required to insert the radioactive seeds into multiple locations in the afflicted tissue, with each needle containing a specified arrangement of the radioactive seeds. Non-radioactive spacers between the radioactive seeds are used to achieve a desired placement of the radioactive seeds specified by the physician's treatment plan.

The implantation needles are accurately located in the prostate gland utilizing a grid template and ultrasound visualization of the implantation needles once they are inserted into the prostate gland. The eventual position of the radioactive seeds and spacers (if utilized) is inferred from the position of the carrier implant needle prior to withdrawal. This procedure is detailed in an article entitled “Ultrasound Guided Transperineal Implantation for the Treatment of Early Stage Prostate Cancer” by Grimm, Blasko, and Ragde, in The Atlas of The UrologicalClinics of North America, Vol. 11, No. 2, October 1994. In 2000, roughly 35% of all men diagnosed with localized prostate gland cancer were treated with radioactive seed implants compared with only about 4% in 1995. Radioactive seed implants have gained widespread acceptance due to the many patient benefits, including long-term results comparable with alternative therapies, for example, radical prostatectomy and external beam radiation therapy without the degree of impotence and incontinence seen following treatment.

In the radioactive seed implant technique, exact positioning of the radioactive seeds is critical to ensuring that the radiation dose delivered to the prostate gland matches the radiation dose prescribed in the physician's treatment plan. As one shortcoming, the radioactive seed implant technique does not prevent the movement of the radioactive seeds in the prostate gland once the implantation needle is removed. Radioactive seeds can migrate within the prostate gland after implantation, and can even move outside the confinement of the prostate gland. As another shortcoming, the initial radioactive seed positioning can be influenced by the technique used to withdraw the implantation needle, whereby the radioactive seeds and spacers are drawn along the implantation needle track as the implantation needle is removed from the prostate gland.

Horowitz (U.S. Pat. No. 4,815,449) describes a radioactive seed delivery system comprising an elongated member made of bioabsorbable material with radioactive seeds dispersed within the elongated member. The elongated member is essentially non-deflecting and is designed for direct insertion into the prostate gland. As one shortcoming, the radioactive seed delivery system does not allow for a variable positioning of the radioactive seeds. As another shortcoming, the radioactive seed delivery system is expensive to realize due to the cost of the process of encapsulating the radioactive seeds within the elongated member.

Grimm (U.S. Pat. Nos. 6,010,446 and 6,450,939) describes spacer elements manufactured from a bioabsorbable material comprising a center section and two cup-like end sections. The cup-like end sections serve to directly hold and receive adjacent radioactive seeds. A series of radioactive seeds and spacer elements form an integral unit which would maintain the relative position of the radioactive seeds in the prostate gland. The spacer elements ensure radioactive seed location following implantation. As one shortcoming, the spacer elements do not provide a leading or trailing end for the series of radioactive seeds and spacer elements.

Thus, a need exists for enhanced spacer elements for implantation seeds.

### SUMMARY

The invention in one embodiment encompasses an apparatus. The apparatus includes a terminus-spacer component that comprises a first end portion and a second end portion. The first end portion is configured to form a coupled connection with an implantation seed. The second end portion serves to terminate a string that comprises one or more spacer components that comprise the terminus-spacer component and one or more implantation seeds that comprise the implantation seed.

Another embodiment of the invention encompasses a method. A terminus-spacer component is employed to terminate a string that comprises one or more implantation seeds and one or more spacer components that comprises the terminus-spacer component. The terminus-spacer component comprises a first end portion that is configured to form a coupled connection with an implantation seed. The terminus-spacer component comprises a second end portion that terminates the string.

### DESCRIPTION OF THE DRAWINGS

Features of exemplary implementations of the invention will become apparent from the description, the claims, and the accompanying drawings in which:

FIG. 1 is a representation of one exemplary implementation of an apparatus that comprises a terminus-spacer component of a string of one or more spacer components and one or more implantation seeds.

FIG. 2 is one exploded representation of the string that comprises the terminus-spacer component of the apparatus of FIG. 1.

FIGS. 3-13 are representations of exemplary configurations of the terminus-spacer component of the apparatus of FIG. 1.

### DETAILED DESCRIPTION

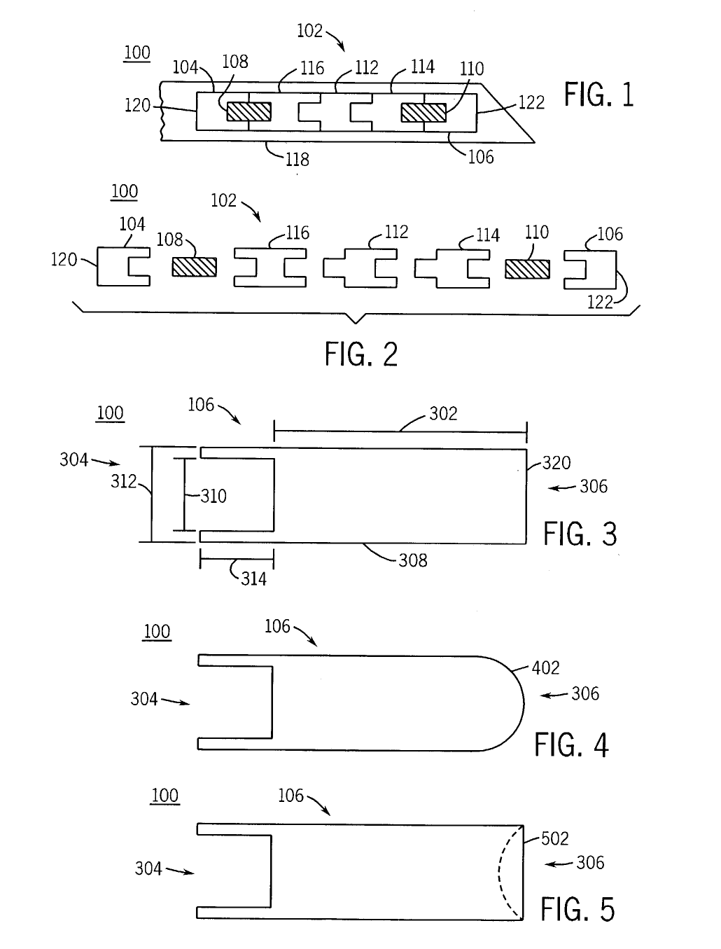

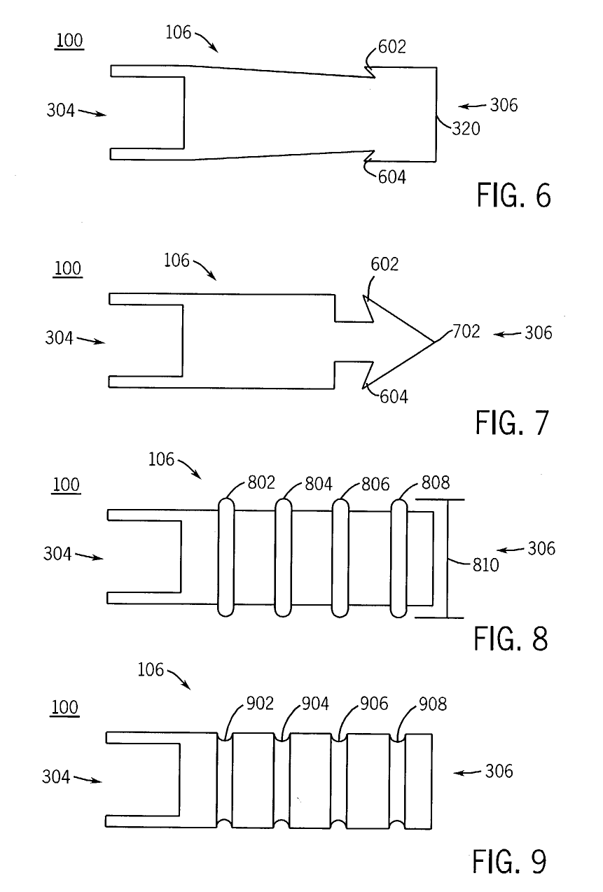

Turning to FIGS. 1 and 2, an apparatus 100 in one example comprises a plurality of components, for example, a terminus-spacer component that comprises a first end portion and a second end portion. The first end portion is configured to form a coupled connection with an implantation seed. The second end portion serves to terminate a string that comprises one or more spacer components that comprise the terminus-spacer component and one or more implantation seeds that comprise the implantation seed. A number of such components can be combined or divided in the apparatus 100.

In one example, the apparatus 100 comprises a string 102 of one or more terminus-spacer components 104 and 106, one or more seeds 108 and 110, one or more intermediary spacer components 112 and 114, and one or more attachment components 116.

The seeds 108 and 110 comprise implantation seeds. The seeds 108 and 110, in one example, comprise radioactive implantation seeds. The seeds 108 and 110 deliver a radiation dose to a tissue, for example, afflicted tissue. In one application, the seeds 108 and 110 deliver a radiation dose to cancer afflicted tissue within a prostate gland. A brachytherapy treatment plan uses the string 102 to administer the radiation dose in accordance with a treatment plan prepared by a physician for a patient. The treatment plan represents the desired distribution pattern for the seeds 108 and 110 in the afflicted tissue. The physician employs medical tools, for example, ultrasound imaging, computerized axial tomography (“CAT”) scans, and X-ray imaging in concert with dose-planning computer software programs for evaluating the medical condition of the patient. Each patient's afflicted tissue varies in size, shape, and location. The present stage of cancer in the afflicted tissue may also vary. Based on the multiple variables of the afflicted tissue and the treatment plan, the physician determines a desired distance of separation for the seeds 108 and 110.

The string 102 serves to establish and maintain a distance of separation between the seeds 108 and 110. The distance of separation is based on a number of the intermediary spacer components 112 and 114 located between the seeds 108 and 110. The string 102 may contain any number and combination of seeds 108 and 110 and intermediary spacer components 112 and 114. The distance of separation may be increased by placing an additional intermediary spacer component substantially similar to the intermediary spacer components 112 and 114 between the seeds 108 and 110. The distance of separation may be decreased by removing one or more of the intermediary spacer components 112 and 114 from between the seeds 108 and 110. The distance of separation is defined by the physician's treatment plan and the string 102 is constructed to achieve the distance of separation. The string 102, in one example, comprises a complete string of the terminus-spacer components 104 and 106, the seeds 108 and 110, the intermediary spacer components 112 and 114, and the attachment component 116. The string 102, in another example, comprises one or more additional intermediary spacer components, seeds, and attachment components substantially similar to the respective intermediary spacer components 112 and 114, the seeds 108 and 110, and the attachment component 116. In the complete string, the one or more additional intermediary spacer components, seeds, and attachment components are between the terminus-spacer components 104 and 106.

To deliver the radiation dose to the afflicted tissue the physician loads the string 102 into an implant needle 118. In one example, the implant needle 118 comprises an eighteen gage implant needle. In another example, the implant needle 118 comprises another implant needle used by the physician. The physician deposits the string 102 from the implant needle 118 into the afflicted tissue in a desired pattern. The seeds 108 and 110 are held in place in the afflicted tissue by the terminus-spacer components 104 and 106, the intermediary spacer components 112 and 114, and the attachment component 116. The terminus-spacer components 104 and 106, the intermediary spacer components 112 and 114, and the attachment component 116 prevent the seeds 108 and 110 from migrating within the afflicted tissue or out of the afflicted tissue.

The terminus-spacer components 104 and 106 provide a spacing distance between one of the seeds 108 and 110 and one of ends 120 and 122 of the string 102. During implantation of the string 102 into the afflicted tissue within the prostate gland, the terminus-spacer components 104 and 106 serve as leading and trailing ends of the string 102. In one example, the terminus-spacer components 104 and 106 comprises one or more anchor components. After implantation of the string 102 into the afflicted tissue within the prostate gland, the anchor components of the terminus-spacer components 104 and 106 inhibit motion of the string 102 in the afflicted tissue within the prostate gland. In another example, the terminus-spacer components 104 and 106 comprises one or more detection promotion components. After implantation of the string 102 into the afflicted tissue within the prostate gland, the detection promotion components of the terminus-spacer component promote an increase of visibility of the terminus-spacer component by an imaging component, for example, ultrasound imaging, computerized axial tomography scans, and X-ray imaging.

The terminus-spacer components 104 and 106 comprise a material that is absorbable in living tissue, for example, bioabsorbable polymers, for example, polylactide, glycolide, caprolactone, polydioxanone, poly (trimethylene carbonate), and copolymers of the bioabsorbable polymers listed herein. Based on the rigidity of the bioabsorbable polymer of the terminus-spacer components 104 and 106, the terminus-spacer components 104 and 106 may be rigid or flexible. Different bioabsorbable polymers may be used to make the terminus-spacer components 104 and 106 more or less rigid or flexible. Structural modifications to the terminus-spacer components 104 and 106, for example, a hollow body within the terminus-spacer components 104 and 106 may make the terminus-spacer components 104 and 106 more or less rigid or flexible.

The intermediary spacer components 112 and 114 allow for a flexibility when constructing the string 102 by allowing for variable spacing. Also, linking the intermediary spacer components 112 and 114 to generate the variable spacing replaces a need to use different sized spacing elements. The intermediary spacer components 112 and 114 comprise a substantially similar design. The intermediary spacer components 112 and 114 are linkable to create a variable sized separator. The variable sized separator maintains a separation between the seeds 108 and 110.

The attachment component 116, in one example, serves to couple a first seed of the seeds 108 and 110 with a second seed of the seeds 108 and 110. The attachment component 116, in another example, serves to couple a first intermediary spacer component of the intermediary spacer components 112 and 114 with a second intermediary spacer component of the intermediary spacer components 112 and 114. The attachment component 116, in yet another example, serves to couple a seed of the seeds 108 and 110 with an intermediary spacer component of the intermediary spacer components 112 and 114.

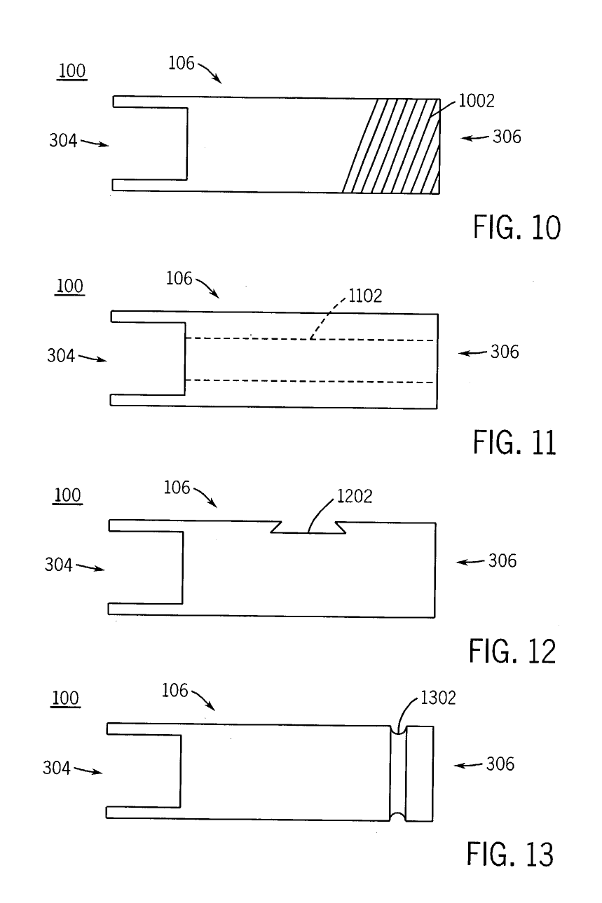

Turning to FIGS. 1 and 3, the terminus-spacer component 106 serves to illustrate one embodiment of the one or more terminus-spacer components 104 and 106. The terminus-spacer component 106 provides a distance of separation corresponding to dimension 302 between the seed 110 and the end 122 of the string 102. In one example, the terminus-spacer component 106 comprises a cylindrical shape. In another example, the terminus-spacer component 106 comprises another shape, such as, a square shape or a hexagonal shape. A largest diameter of the terminus-spacer component 106 is small enough to enable the termninus-spacer component 106 to pass through the implant needle 118, for example, the eighteen gage implant needle. The terminus-spacer component 106 comprises end portions 304 and 306 and a center portion 308.

The end portion 304 of the terminus-spacer component 106 is configured to form a coupled connection with any one of the seeds 108 and 110, and the intermediary spacer components 112 and 114. For example, the end portion 304 receives and holds any one of the seeds 108 and 110, and the intermediary spacer components 112 and 114. The end portion 304 comprises a cup-like configuration. For example, the end portion 304 comprises an inner diameter 310, an outer diameter 312, and a dimension 314.

The inner diameter 310 of the end portion 304 is substantially similar to or slightly larger than a diameter of the seeds 108 and 110. The inner diameter 310 of the end portion 304 is substantially similar to or slightly larger than a mating diameter of the intermediary spacer components 112 and 114. Therefore, the inner diameter 310 may receive and hold any one of the seeds 108 and 110, and the intermediary spacer components 112 and 114. The inner diameter 310 typically corresponds to the diameter of one of the seeds 108 and 110, for example, a standard size of the seeds 108 and 110 is about 0.8 millimeters. Therefore, the inner diameter 310 is able to receive and hold the seeds 108 and 110. However, the inner diameter 310 may be any size that allows the end portion 304 to receive and hold a seed of any size. The size of the inner diameter 310 is relative to a strength of the attachment between the terminus-spacer component 106 and any one of the seeds 108 and 110, and the intermediary spacer components 112 and 114. For a tighter fit and a stronger attachment, the inner dimension 310 may be slightly reduced from the standard size of the seeds 108 and 110. For a looser fit and a weaker attachment, the inner dimension 310 may be slightly increased from the standard size of the seeds 108 and 110.

The outer diameter 312 of the end portion 304 is small enough to enable the end portion 304 to pass through the implant needle 114. In one example where the implant needle 114 comprises the eighteen gage implant needle, the outer diameter 312 is typically about one millimeter to allow the terminus-spacer component 106 to pass through the eighteen gage implant needle. However, the outer diameter 312 may be any size that allows the terminus-spacer component 106 to pass through any size implant needle.

The dimension 314 of the end portion 304 corresponds to a depth of the cup-like configuration. The dimension 314 may be between 0.1 and 0.2 centimeters. However, different values of the dimension 314 will achieve different levels of strength in the attachment between the terminus-spacer component 106 and any one of the seeds 108 and 110, and the intermediary spacer components 112 and 114. Alternatively, the end portion 304 comprises a flared receptacle to facilitate connection with any one of the seeds 108 and 110, and the intermediary spacer components 112 and 114. The end portion 304 may be otherwise altered to facilitate holding any one of the seeds 108 and 110, and the intermediary spacer components 112 and 114.

The end portion 306 of the terminus-spacer component 106 serves to terminate the string 102. The end portion 306 provides a leading or trailing end to the string 102. The end portion 306 comprises a blunt circular end surface 320 of the cylindrically shaped terminus-spacer component 106. The blunt circular end surface 320 promotes a reduction in mobility of the string 102. The blunt circular end surface 320 comprises a sharp reflective edge. The sharp reflective edge promotes an increase in visibility of the terminus-spacer component 106 by the imaging component, for example, ultrasound imaging. The blunt circular end surface 320 provides a flat surface to accept pressure from a stylet of the implant needle 118.

The center portion 308 of the terminus-spacer component 106 provides the distance of separation corresponding to the dimension 302. In one example, the distance of separation that corresponds to the dimension 302 is between 0.5 and 1.0 centimeters. In another example, the distance of separation corresponding to the dimension 302 is any size within the requirements of the treatment plan. In one example, the center portion 308 comprises a solid bioabsorbable material. In another example, the center portion 308 comprises one or more cavities or hollow portions.

Turning to FIGS. 1 and 4, the terminus-spacer component 106 serves to illustrate one embodiment of the one or more terminus-spacer components 104 and 106. The terminus-spacer component 106 of FIG. 4 is analogous to the terminus-spacer component 106 of FIG. 3 with the exception of the end portion 306. The end portion 306 comprises a convex rounded end surface 402 of the cylindrically shaped terminus-spacer component 106. The convex rounded end surface 402, in one example, emulates a rounded end profile of the seeds 108 and 110. The convex rounded end surface 402 promotes a smooth passage through the afflicted tissue.

Turning to FIGS. 1 and 5, the terminus-spacer component 106 serves to illustrate one embodiment of the one or more terminus-spacer components 104 and 106. The terminus-spacer component 106 of FIG. 5 is analogous to the terminus-spacer component 106 of FIG. 3 with the exception of the end portion 306. The end portion 306 comprises a concave rounded end surface 502 of the cylindrically shaped terminus-spacer component 106. The concave rounded end surface 502 promotes a reduction in mobility of the string 102. The concave rounded end surface 502 comprises a sharp reflective edge. The sharp reflective edge promotes an increase in visibility of the terminus-spacer component 106 by the imaging component, for example, ultrasound imaging. The concave rounded end surface 502 provides a mating surface pocket to accept pressure from the stylet of the implant needle 118.

Turning to FIGS. 1 and 6-7, the terminus-spacer component 106 serves to illustrate one embodiment of the one or more terminus-spacer components 104 and 106. The terminus-spacer component 106 of FIGS. 6 and 7 are analogous to the terminus-spacer component 106 of FIG. 3 with the addition of one or more barbs 602 and 604. The one or more barbs 602 and 604 serve to anchor the string 102 into the afflicted tissue within the prostate gland therefore limiting a movement of the seeds 108 and 110 in the afflicted tissue within the prostate gland. The terminus-spacer component 106 of FIG. 6 comprises the blunt circular end surface 320. The terminus-spacer component 106 of FIG. 7 comprises a pointed end 702 as another modification to the terminus-spacer component 106 of FIG. 3.

Turning to FIGS. 1 and 8, the terminus-spacer component 106 serves to illustrate one embodiment of the one or more terminus-spacer components 104 and 106. The terminus-spacer component 106 of FIG. 8 is analogous to the terminus-spacer component 106 of FIG. 3 with the addition of one or more ribs 802, 804, 806, and 808. The one or more ribs 802, 804, 806, and 808 serve to anchor the string 102 into the afflicted tissue within the prostate gland therefore limiting a movement of the seeds 108 and 110 in the afflicted tissue within the prostate gland. An outer diameter 810 of the terminus-spacer component 106 with the one or more ribs 802, 804, 806, and 808 is small enough to enable the terminus-spacer component 106 to pass through the implant needle 118. The one or more ribs 802, 804, 806, and 808 serve to enhance the visibility of the terminus-spacer component 106 by the imaging components, for example, ultrasound imaging.

Turning to FIGS. 1 and 9, the terminus-spacer component 106 serves to illustrate one embodiment of the one or more terminus-spacer components 104 and 106. The terminus-spacer component 106 of FIG. 9 is analogous to the terminus-spacer component 106 of FIG. 3 with the addition of one or more grooves 902, 904, 906, and 908. The one or more grooves 902, 904, 906, and 908 serve to anchor the string 102 into the afflicted tissue within the prostate gland therefore limiting a movement of the seeds 108 and 110 in the afflicted tissue within the prostate gland. The one or more grooves 902, 904, 906, and 908 serve to enhance the visibility of the terminus-spacer component 106 by the imaging components, for example, ultrasound imaging.

Turning to FIGS. 1 and 10, the terminus-spacer component 106 serves to illustrate one embodiment of the one or more terminus-spacer components 104 and 106. The terminus-spacer component 106 of FIG. 10 is analogous to the terminus-spacer component 106 of FIG. 3 with the addition of a screw threading 1002 to the end 306. The screw threading 1002 serves to anchor the string 102 into the afflicted tissue within the prostate gland therefore limiting a movement of the seeds 108 and 110 in the afflicted tissue within the prostate gland. The screw threading 1002 may also connect with a mating screw threading of a control component. The control component may provide direct control of the string 102. The screw threading 1002 may comprise any of raised threading, recessed threading, barb, bayonet-type fitting, sleeve, and connection component that mates with the control component.

Turning to FIGS. 1 and 11, the terminus-spacer component 106 serves to illustrate one embodiment of the one or more terminus-spacer components 104 and 106. The terminus-spacer component 106 of FIG. 11 is analogous to the terminus-spacer component 106 of FIG. 3 with the addition of a cavity 1102 encapsulated within the terminus-spacer component 106. The cavity 318 may comprise one or more individual cavities or may pass completely through the terminus-spacer component 106. The cavity 318 promotes detection of the terminus-spacer component 106 by an imaging component, for example, ultrasound imaging, computerized axial tomography scans, and X-ray imaging. A portion of the cavity 318 may contain contrast agents to additionally promote detection of the terminus-spacer component 106 by the imaging component, for example, one or more of ultrasound contrast agents, gadolinium, gadolinium salts, X-ray markers, air pockets, electronic sensors, and microchips. The contrast agents enhance the visibility of the terminus-spacer component 106 by the imaging component during implantation. The contrast agents also enhance the visibility of the terminus-spacer component 106 to promote location of the terminus-spacer component 106 after implantation. In other examples, the contrast agents are contained in a recessed pocket 1202 (FIG. 12) or a groove 1302 (FIG. 13).

The steps or operations described herein are just exemplary. There may be many variations to these steps or operations without departing from the spirit of the invention. For instance, the steps may be performed in a differing order, or steps may be added, deleted, or modified.

Although exemplary implementations of the invention have been depicted and described in detail herein, it will be apparent to those skilled in the relevant art that various modifications, additions, substitutions, and the like can be made without departing from the spirit of the invention and these are therefore considered to be within the scope of the invention as defined in the following claims.

What is claimed is:

1. An apparatus, comprising:
a terminus-spacer component that comprises a first end portion and a second end portion;
wherein the first end portion is configured to form a coupled connection with an implantation seed;
wherein the second end portion serves to terminate a string that comprises one or more spacer components that comprise the terminus-spacer component and one or more implantation seeds that comprise the implantation seed.

2. The apparatus of claim 1, wherein the first end portion of the terminus-spacer component comprises a cup configuration, wherein the cup configuration of the terminus-spacer component comprises a mating relationship with an end portion of the implantation seed.

3. The apparatus of claim 1, wherein the first end portion of the terminus-spacer component is configured to form a coupled connection with an intermediary spacer component of the one or more spacer components.

4. The apparatus of claim 3, wherein the first end portion of the terminus-spacer component comprises a cup configuration, wherein the cup configuration of the terminus-spacer component comprises a mating relationship with an end portion of the intermediary spacer component.

5. The apparatus of claim 1, wherein during implantation the string comprises a lead end and a trail end, wherein the terminus-spacer component serves to terminate the string at the lead end of the string.

6. The apparatus of claim 5, wherein the terminus-spacer component comprises a first terminus-spacer component, wherein a second terminus-spacer component serves to terminate the string at the trail end of the string.

7. The apparatus of claim 1, wherein during implantation the string comprises a lead end and a trail end, wherein the terminus-spacer component serves to terminate the string at the trail end of the string.

8. The apparatus of claim 1, wherein during implantation the string comprises a lead end and a trail end, wherein the terminus-spacer component comprises a dimension that contributes to a distance of separation between the implantation seed and the lead end or the trail end.

9. The apparatus of claim 1, wherein the second end portion of the terminus-spacer component comprises a blunt end.

10. The apparatus of claim 1, wherein the second end portion of the terminus-spacer component comprises a convex end.

11. The apparatus of claim 1, wherein the second end portion of the terminus-spacer component comprises a concave end.

12. The apparatus of claim 1, wherein the terminus-spacer component comprises one or more barbs that serve to anchor the string.

13. The apparatus of claim 1, wherein the terminus-spacer component comprises one or more raised ribs that serve to anchor the string.

14. The apparatus of claim 1, wherein the terminus-spacer component comprises one or more grooves that serve to anchor the string.

15. The apparatus of claim 1, wherein the terminus-spacer component comprises a screw thread that serves to anchor the string.

16. The apparatus of claim 1, wherein the terminus-spacer component comprises a cavity that serves to promote detection of the terminus-spacer component by an imaging component.

17. The apparatus of claim 16, wherein a portion of the cavity is filled with a contrast agent, wherein the contrast agent promotes detection of the terminus-spacer component by the imaging component.

18. The apparatus of claim 1, wherein the terminus-spacer component comprises a recessed pocket, wherein a portion of the recessed pocket is filled with a contrast agent, wherein the contrast agent promotes detection of the terminus-spacer component by an imaging component.

19. The apparatus of claim 1, wherein a largest diameter of the terminus-spacer component is small enough to enable the terminus-spacer component to pass through an implant needle.

20. The apparatus of claim 19, wherein the largest diameter of the terminus-spacer component is small enough to enable the terminus-spacer component to pass through an eighteen gage implant needle.

21. The apparatus of claim 1, wherein the terminus-spacer component comprises a material that is absorbable by living tissue.

22. The apparatus of claim 1, wherein the terminus-spacer component serves as a terminus to the string that comprises the one or more spacer components and the one or more implantation seeds that comprise of one or more radioactive seeds.

23. The apparatus of claim 22, wherein string that comprises the terminus-spacer component and the one or more radioactive seeds serves to deliver a dose of radiation to afflicted tissue of a prostate gland.

24. A method, comprising the step of:
employing a terminus-spacer component to terminate a string that comprises one or more implantation seeds and one or more spacer components that comprises the terminus-spacer component;
wherein the terminus-spacer component comprises a first end portion that is configured to form a coupled connection with an implantation seed;
wherein the terminus-spacer component comprises a second end portion that terminates the string.

25. The method of claim 24, further comprising the step of:
forming a mating relationship between a cup configuration of the first end portion of the terminus-spacer component and an end portion of the implantation seed.

26. The method of claim 24, wherein the one or more implantation seeds of the string comprise one or more radioactive seeds, further comprising the steps of:
loading the string into an implantation needle;
implanting the string into afflicted tissue within a prostate gland; and
delivering a radiation dose from the radioactive seeds of the string to the afflicted tissue within the prostate gland.

27. The method of claim 26, further comprising the step of:
inhibiting motion of the string in the afflicted tissue within the prostate gland with one or more anchor components of the terminus-spacer component.
